(19) United States
(12) Patent Application Publication
(10) Pub. No.: US 2005/0271585 A1
(43) Pub. Date: Dec. 8, 2005

(54) Extended conjugated polymers

(75) Inventors: Egidijus Edward Uzgiris, Schenectady, NY (US); Mohan Mark Amaratunga, Clifton Park, NY (US)

Correspondence Address: GENERAL ELECTRIC COMPANY;GLOBAL RESEARCH, PATENT DOCKET RM. BLDG. K1-4A59, NISKAYUNA, NY, 12309, US

(73) Assignee: General Electric Company

(21) Appl. No.: 10/861,879

(22) Filed: Jun. 7, 2004

(51) Int. Cl. A61K 51/00; A61K 49/00

(52) U.S. Cl. 424/1.11; 424/9.323

(57) ABSTRACT

A polymer is conjugated to a plurality of molecules having at least a functionality and further comprises an active agent, such as a diagnostic agent or a therapeutic agent. The diagnostic agent is capable of generating a signal detectable by a medical imaging technique. The conjugated polymer has an extended conformation and provides enhanced contrast of images of diseased tissues or enhanced delivery of therapeutic agents to these tissues.

### BACKGROUND OF THE INVENTION

The present invention relates to conjugated polymers having an extended conformation. In particular, the present invention relates to poly(amino acid) polymers being conjugated to active agents and having an extended conformation.

In many medical procedures it is important to accumulate a certain active agent to a desired tissue type. For example, in chemotherapy, it is important to deliver drugs only to cancerous tumor tissue, and not to normal tissue, since these drugs destroy the tissue with which they come in contact. Another example would be in medical imaging. Contrast agents are attached to carrier molecules, which are specific to tumor tissue. As the carrier molecules concentrate in the tumor tissue, the contrast agents enhance a medical image of this tissue.

The use of a chemotherapy drug (e.g., Doxorubicin) attached to Poly-L-aspartic acid (“PAA”) has been previously described. Many of the carrier molecules employed are proteins having a globular or folded configuration.

One known type of carrier molecule contains polypeptides having a diameter larger than pores of blood vessels of normal tissue and smaller than pores of blood vessels of tumor tissue. See U.S. Pat. No. 5,762,909. Carriers of this type have a length several orders of magnitude greater than their diameter, a net negative charge, and have an extended or elongated chain conformation with a long persistence length. These carriers move about in a worm-like manner. Lanthanide complexes (e.g., gadolinium-diethylenetriamine pentaacetic acid complexes) are attached to these carrier molecules to create complex molecules, which are introduced into a blood vessel of the subject.

These complex molecules pass though the pores of only the tumor endothelium and interact with the fibrous structures of the tumor interstitium. The penetration of the tumor interstitium by the complex molecules is enhanced by the worm-like configuration of the complex molecule which allows the molecule to “snake” around fixed obstacles in the extracellular matrix of the tumor interstitium.

In order to achieve a worm-like configuration, when the polylysine disclosed in U.S. Pat. No. 5,762,909 or in other prior-art references is conjugated to diethylenetriamine pentaacetic acid (DTPA) molecules, ninety percent or more of the lysine residues must be attached to DTPA molecules to eliminate or reduce intra-chain ionic bonds as well to allow charge repulsion between DTPA moieties to unfold and extend the polymer chain. The amount of substitutions (also referred to as the degree of conjugation) thus affects the configuration of the resulting complex, with a higher degree of conjugation providing a more consistent extended structure and better targeting. Unfortunately, it is difficult to reliably attain degrees of conjugation of higher than 90 percent. (See Sieving et al., Bioconjugate Chem., Vol. 1, 65 (1990).) Substitutions of above 90 percent are as rare as 1 in 7 synthesis runs, even with high anhydride to lysine ratios and extended reaction times. Yet, this level of substitution is required for the proper polymer configuration to be realized in the case of this homopolymer.

Therefore, there is a continued need to provide improved conjugated polymers that have extended conformation. In addition, it is very desirable to provide such conjugated polymers the extended conformation of which is more consistently achieved than those of the prior art.

### SUMMARY OF THE INVENTION

In general, the present invention provides conjugated polymers that have extended conformation.

According to one aspect of the present invention, a conjugated polymer comprises a poly(amino acid) backbone, a plurality of amino acid residues of the backbone is conjugated to molecules having at least a functionality such that the conjugated poly(amino acid) achieves an extended conformation.

According to another aspect, the molecule having at least a functionality is poly(carboxylic acid) molecule.

According to still another aspect, the poly(carboxylic acid) molecule is p-isothiocyanatobenzyl-1,4,7,10-tetraazacyclododecane-1,4,7,10-tetraacetic acid (“p-SCN-Bz-DOTA”).

According to still another aspect, the degree of conjugation of the amino acid residues is at least about 50 percent. The term “degree of conjugation” with respect to a type of amino acid residues of the backbone chain means the percentage of that type of amino acid residues that are conjugated to the molecules having said at least a functionality.

According to yet another aspect, the degree of conjugation is in the range from about 50 percent to about 98 percent.

According to yet another aspect, the extended conjugated polymer further comprises an active agent that is associated with the conjugated molecules having said at least a functionality or that is linked to extended conjugated polymer.

Other features and advantages of the present invention will be apparent from a perusal of the following detailed description of the invention and the accompanying drawings in which the same numerals refer to like elements.

### BRIEF DESCRIPTION OF THE DRAWINGS

FIG. 1 is an illustration of inter-chain and intra-chain attraction of polypeptides.

FIG. 2 is an illustration of a highly conjugated polypeptide of the present invention.

FIG. 3 shows circular dichroism spectra of polylysine conjugated to DTPA that binds gadolinium ions having a degree of conjugation of ______, and of polylysine conjugated to p-SCN-Bz-DOTA that binds gadolinium ions having a degree of conjugation of ______.

FIG. 4 shows retention times of various polymeric materials using HPLC separation.

FIG. 5 shows MRI signal changes in tumor tissues upon administering polylysine conjugated to DOTA that binds Gd³⁺ ions and polylysine conjugated to p-SCN-Bz-DOTA that binds Gd³⁺ ions.

### DETAILED DESCRIPTION OF THE PREFERRED EMBODIMENT

In general, the present invention provides conjugated polymers that have extended conformation.

According to one aspect of the present invention, a conjugated polymer comprises a poly(amino acid) backbone, a plurality of amino acid residues of the backbone is conjugated to molecules having at least a functionality such that the conjugated poly(amino acid) achieves an extended conformation. In the present disclosure, the terms “poly(amino acid)” and “polypeptide” are used interchangeably. The term “contrast-enhancing agent” is sometimes abbreviated to “contrast agent.”

Conformation of the Conjugated Polymer

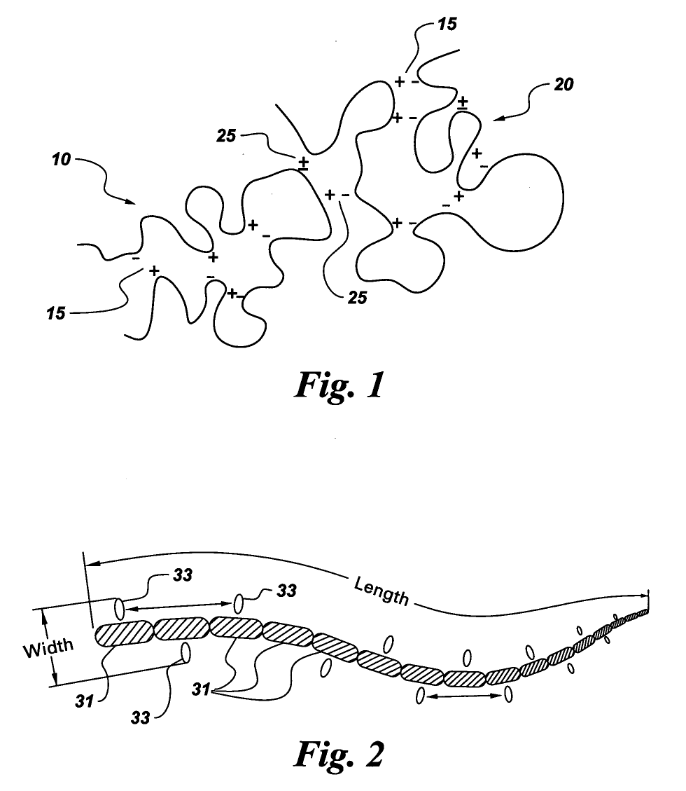

An extended conjugated polymer, such as a poly(amino acid), of the present invention has an elongated, worm-like conformation. In general, the conformation of a polymer is a result of interaction of intra-chain charges, which interaction is manifested in the extent of rigidity of the polymer molecule. In general, poly(amino acid) molecules in solution carry opposite charges at the amino and carboxylic acid groups, which interact with each other often to result in a bulky tightly folded or globular conformation. For example, FIG. 1 illustrates two poly(amino acid) chains 10 and 20, each carrying a plurality of positive and negative charges. The segments of the same poly(amino acid) chain 10 or 20 carrying opposite charges attract to each other at 15, resulting in highly folded chains. In addition, opposites charges carried on adjacent chains 10 and 20 also attract to each other at 25, resulting in the formation of large globules, each of which comprises a plurality of chains. On the other hand, residues of a poly(amino acid) chain of the present invention is conjugated to molecules having at least a functionality, such as chelator moieties having net negative charges that inhibit the attraction between segments of the chain so as to result in an elongated conformation. The degree of conjugation of a poly(amino acid) chain of the present invention is at least about 50 percent, preferably from about 50 percent to about 98 percent. By “conjugation” or “conjugated,” it is meant in this disclosure that an amino acid residue of the poly(amino acid) chain is attached covalently with at least a portion of another molecule having at least a functionality. In one embodiment, this molecule having said at least a functionality is a chelator capable of binding or localizing a cation. Thus, the process of conjugation also includes a process of substitution of at least one atom of an amino acid residue with a portion of the chelator. (The term “residue”, as used in this disclosure, means the remaining portion of a monomeric unit that is linked with portions of other monomeric units to form the polymer.) FIG. 2 illustrates a poly(amino acid) chain comprising amino acid residues 31 linked together through peptide bonds. Each residue 31 of a fraction (from about 50 percent to about 98 percent) is conjugated to chelator 33 through a covalent bond. Chelators 33 inhibits, by steric hindrance and charge repulsion, the tendency of the poly(amino acid) to become folded upon itself, resulting a stretched out conformation. Therefore, a conjugated poly(amino acid) of the present invention can easily enter small pores or spaces, such as a porous space between endothelial cells in an atherosclerotic region of a blood vessel or the many small blood vessels typically present at tumor tissues, but at the same time is not easily cleared from the body of the subject. Persistence length is a measure that can quantify the “straightness” of a polymeric chain and is a useful parameter characterizing a contrast-enhancing agent of the present invention. Persistence length is the average projection of the end-to-end distance vector (the vector connecting the two ends of the polymer molecule) on the direction of a selected bond vector. The persistence length can be calculated using the radius of gyration of the polymer molecule, which radius of gyration can be determined by a light scattering experiment. See; e.g., Charles R. Cantor and Paul R. Schimmel, “Biophysical Chemistry, Part III: The Behavior of Biological Macromolecules,” pp. 979-1018, W.H. Freeman and Company, New York, N.Y. (1980); Charles R. Cantor and Paul R. Schimmel, “Biophysical Chemistry, Part II: Techniques for the Study of Biological Structure and Function,” pp. 838-846, W.H. Freeman and Company, New York, N.Y. (1980); and Paul J. Flory, “Statistical Mechanics of Chain Molecules,” pp. 36-38, Oxford University Press, New York, N.Y. (1989). The cited sections of these references are incorporated herein by reference. A contrast-enhancing agent of the present invention has a worm-like shape being essentially a stretched-out, extended chain with little folding. A folded poly(amino acid) with little or no conjugation, has a low persistence length of about 10 angstroms, and is not suitable for use in the present invention. On the other hand, a conjugated polymer of the present invention has a persistence length in the range from about 100 to about 600 angstroms. The backbone chain of a conjugated polymer of the present invention typically has from about 50 to about 1500, preferably from about 100 to about 650, monomeric amino acid residues.

The conformation of poly(amino acid) chains is also discussed in U.S. Pat. No. 5,762,909; which is incorporated in its entirety in the present disclosure by reference.

In one approach to produce an effective conjugated polymer complex having a proper persistence length, one eliminates or reduces intra-chain charge interactions as well as restricts rotation about a bond at each peptide link. This may be accomplished by making substitution of the chain with a molecule that provides a steric hindrance, extending as side arms from the main chain.

For example, if the polypeptide backbone chain is poly-L-lysine (“PLL”), which has a positive charge at each lysine, one attaches a sufficient amount of substitutions that would impair peptide bond rotation. When PLL is conjugated with DTPA, at least about ninety percent of the lysine residues must be conjugated in order to achieve an extended conformation. See U.S. Pat. No. 5,762,909.

The present inventors unexpectedly discovered that when a polypeptide backbone chain that has positive charges at a substantial portion of the chain is conjugated to p-isothiocyanatobenzyl-1,4,7,10-tetraazacyclododecane-1,4,7,10-tetraacetic acid (“p-SCN-Bz-DOTA”), the resulting conjugated polypeptide can adopt an extended conformation with a degree of conjugation less than 90 percent. One such polypeptide is polylysine, which is suitable for conjugation to p-SCN-Bz-DOTA. Polylysine conjugated to p-SCN-Bz-DOTA can adopt an extended conformation with a degree of conformation of about 75 percent. Polylysine conjugated to p-SCN-Bz-DOTA usable for a method of detecting or treating a diseased tissue can have a degree of conjugation between about 50 percent and about 75 percent.

Other polypeptides that can be conjugated to p-SCN-Bz-DOTA are polyhistidine, polyarginine, polyasparagine, polyglutamine, and copolymers of at least two types of monomeric units selected from the group consisting of lysine, histidine, and arginine. Other polypeptides that can benefit from a conjugation with p-SCN-Bz-DOTA to produce a compound of the present invention having an elongated conformation comprise one or more types of monomeric units of lysine, histidine, arginine, asparagine, and glutamine. Other copolymers suitable for the present invention comprise at least a first type of monomeric units selected from the group consisting of histidine, arginine, asparagine, and glutamine, and at least a second type of monomeric units selected from the group consisting of glutamic acid and aspartic acid.

Diagnostic Imaging Contrast-Enhancing Agents

In one embodiment of the present invention, a contrast-enhancing agent for used in diagnostic imaging of a portion of a subject comprises a polymeric backbone conjugated to a plurality of molecules having a functionality. The conjugated polymer has a substantially extended conformation. The plurality of molecules having a functionality further binds an active agent that can generate a signal detectable by an imaging technique.

In one embodiment, the polymeric backbone is selected from the group consisting of homopolymers and copolymers disclosed herein above. Preferably, the polymeric backbone is polylysine having a number of lysine residues in the range from about 50 to about 1500. More preferably, the number of lysine residues is in the range from about 100 to about 650.

In another aspect the molecule having a functionality is p-SCN-Bz-DOTA, which is conjugated to a lysine residue at its free amino side group. Such conjugation is effected, for example, through the isothiocyanato group on p-SCN-Bz-DOTA. See; e.g., K. G. Mann and W. W. Fish, “Protein Polypeptide Chain Molecular Weights by Gel Chromatography in Guanidinium Chloride,” Methods in Enzymology, Vol. 26, pp 28-42 (1972); J. E. Sinsheimer et al., “Fluorescein Isothiocyanates: Improved Synthesis and Purity Spectral Studies,” Anal. Biochem., Vol. 57, pp 227-231 (1974).

In another embodiment, the active agent is a paramagnetic ion that is capable of enhancing the contrast of images acquired in the magnetic resonance imaging (“MRI”) technique, such as gadolinium ion or dysprosium ion.

In still another embodiment, the active agent is Cu-64 ion that is capable of generating positron emission detectable by the positron emission tomography (“PET”) technique. In this case, it may be adequate to have such PET active agent bound to one or a few, such as fewer than 20, of the conjugated p-SCN-Bz-DOTA moieties. It may be adequate to have fewer than 10 of the conjugated p-SCN-Bz-DOTA moieties bind the PET active agent.

In yet another embodiment, the PET active agent can be a radioactive-iodine (such as I-124) or radioactive-fluorine labeled moiety, which is covalently attached to a free amino group of a lysine residue. Non-limiting examples of such radioactive labeled moiety are 4-iodobenzamide labeled with I-124 and 4-fluorobenzamide labeled with F-18. Such a PET contrast-enhancing agent can be manufactured from poly(amino acid) backbone chains comprising lysine and at least an amino acid other than lysine. In one embodiment, such other amino acid is selected from among those disclosed herein above.

Therapeutic Agents

In one embodiment of the present invention, a therapeutic agent for use in treating a diseased tissue in a subject comprises a polymeric backbone conjugated to a plurality of molecules having a functionality. The conjugated polymer has a substantially extended conformation. The plurality of molecules having a functionality further binds an active agent that can produce a beneficial effect on the diseased tissue. The beneficial effect can be, for example, killing cells of the diseased tissue.

In one embodiment, the polymeric backbone is selected from the group consisting of homopolymers and copolymers disclosed herein above. Preferably, the polymeric backbone is polylysine having a number of lysine residues in the range from about 50 to about 1500. More preferably, the number of lysine residues is in the range from about 100 to about 650.

In another aspect the molecule having a functionality is p-SCN-Bz-DOTA, which is conjugated to a lysine residue at its free amino side group. Such conjugation is effected, for example, through the isothiocyanato group on p-SCN-Bz-DOTA.

Among the therapeutic agents useful in the current invention are radioisotopes, drugs, toxins, fluorescent dyes activated by nonionizing radiation, hormones, hormone antagonists, receptor antagonists, enzymes or proenzymes activated by another agent, autocrine, or cytokine. Many drugs and toxins are known which have cytotoxic effects on cells. They can be found in compendia of drugs and toxins, such as the Merck Index, Goodman and Gilman's “The Pharmacological Basis of Therapeutics” (Tenth Edition, McGraw-Hill, New York, 2001).

Radioisotopes of metals for therapeutic use include: actinium-211, actinium-225, bismuth-212, bismuth-213, lead-212, lead 203, rhenium-186, rhenium-188, silver-111, platinum-197, palladium-109, ruthenium-97, copper-67, copper-64, yttrium-90, scandium-47, samarium-153, lutetium-177, rhodium-105, praseodymium-142, praseodymium-143, terbium-161, holmium-166, gold-199, technicium-99m, indium-111, indium-113m, gallium-67, and gallium-68. These radioisotopes may be bound to the p-SCN-Bz-DOTA moieties, which are conjugated to the polypeptide backbone.

Other radioisotopes, such as I-125 or I-131, may be provided by covalently attaching 4-iodobenzamide, in which the iodine atom is I-125 or I-131, to a free amino group of a lysine residue. Benzamide may also be labeled with Br-76, Br-77, or At-211 and similarly attached to the free amino group of a lysine residue.

Other therapeutic agents of an organic nature (e.g., drugs, toxins, hormones, enzyme, proenzymes, receptor antagonists, cytokine, etc.) can be conjugated, coupled, or otherwise attached to the extended polypeptide of the present invention at one or more amino acid residues by conventional means and/or chemistry well known in the art. For example, such conjugation or attachment may be effected through a reaction with the free amino group of a lysine residue, or with a thiol group of cysteine residues introduced into the polypeptide backbone.

### EXAMPLE

### Preparation of Polylysine Conjugated to p-SCN-Bz-DOTA That Chelates Gadolinium Ions

pSCN-Bz-DOTA.4HCl was purchased from Macrocyclics (Dallas, Tex.). Poly-L-lysine hydrobromide was purchased from Sigma (St. Louis, Mo.), having a degree of polymerization of 402. An aqueous solution of pSCN-Bz-DOTA.4HCl (500 mg, 0.72 mmol, 48 mM) was added to a solution of 25 mg of poly-L-lysine hydrobromide (25 mg, 12 mM) in 0.1 M tetramethylammonium phosphate buffer (pH of 9) at room temperature, with stirring. Initial pH dropped to 2.2. Over a 30-minute period, additional buffer (5 ml) and aqueous 2.0M triethanolamine (5 ml) were added as needed to adjust pH to 8.5-9.0. After 30 minutes, the pH remained unchanged and solution was stirred at room temperature for 16 hours. Purification was by diafiltration.

Gadolinium ions were bound to the p-SCN-Bz-DOTA-conjugated polylysine thus produced by contacting such conjugated polylysine with

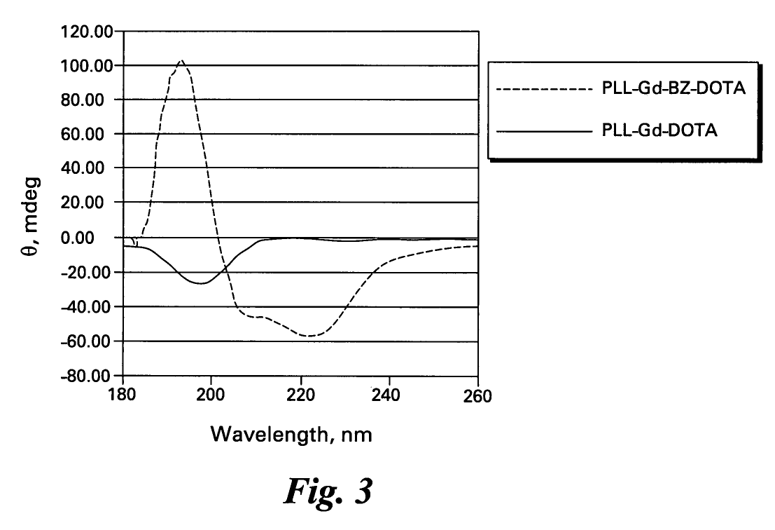

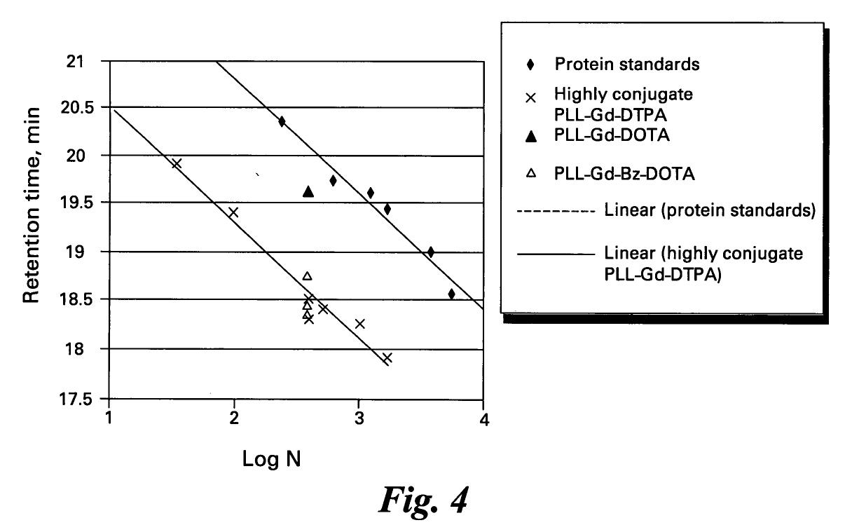

FIG. 3 shows circular dichroism (“CD”) spectra of p-SCN-Bz-DOTA-conjugated polylysine and DOTA-conjugated polylysine, both chelated with gadolinium ions. The positive peak in the wavelength range of about 190-200 nm in the CD spectrum of p-SCN-Bz-DOTA-conjugated polylysine is characteristic of an extended polymer. Such feature is absent in the CD spectrum of DOTA-conjugated polylysine. Further evidence that p-SCN-Bz-DOTA-conjugated polylysine has a more extended conformation than DOTA-conjugated polylysine or protein standards that are known to have more coiled and globular conformation is shown in FIG. 4, which shows HPLC retention time versus logarithm of the number of amino acid residues. HPLC retention time is shorter for more extended polymers for a given number of amino acid residues. The p-SCN-Bz-DOTA-conjugated polylysine samples of the present invention have extended conformation similar to that of DTPA-conjugated polylysine having degrees of conjugation greater than 90 percent.

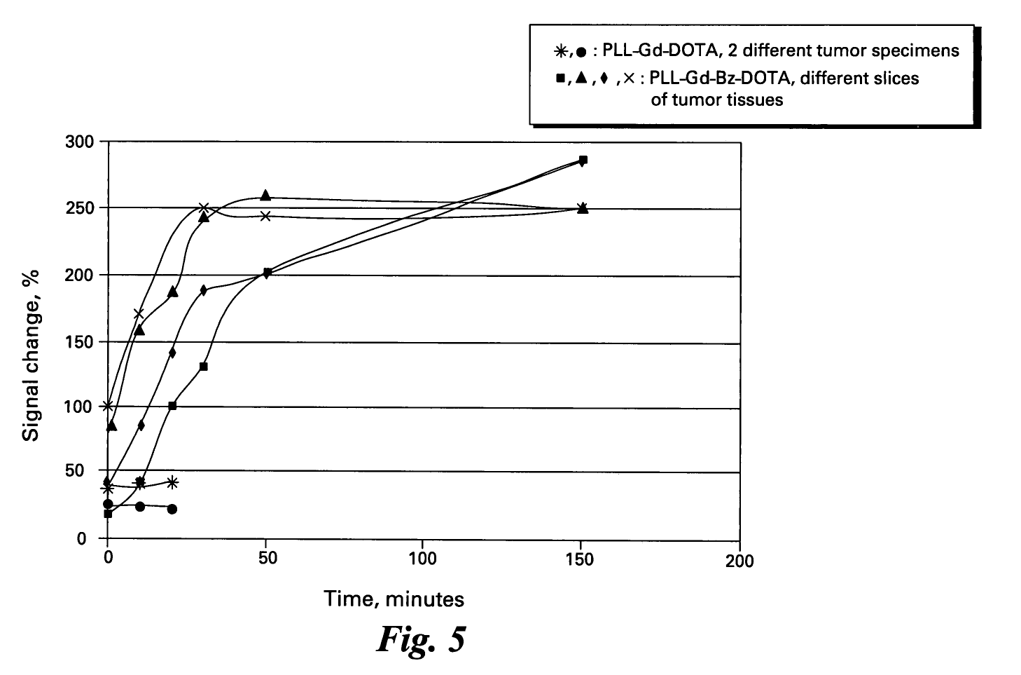

Changes in MRI signals from tumor tissues of animal models are shown in FIG. 5, which shows that p-SCN-Bz-DOTA-conjugated polylysine cross the endothelium of the tumor and accumulate there more rapidly than DOTA-conjugated polylysine.

### METHOD FOR DETECTING OR TREATING A DISEASED TISSUE USING AN EXTENDED CONJUGATED POLYMER OF THE PRESENT INVENTION

The present invention provides a method for detecting, assessing, or treating a diseased tissue or a portion of a subject using an extended conjugated polymer disclosed herein. The method of the present invention, for detecting or assessing a diseased tissue, can employ one or more medical imaging techniques, such as MRI, PET, Computed Tomography (“CT”), Single Photon Emission Computed Tomography (“SPECT”), X-ray imaging, etc. The method of the present invention also allows for assessing an effectiveness of a prescribed regimen for treating a diseased tissue. For example, the state of tumor tissues can be assessed by imaging the formation of blood vessels in the tissues using MRI contrast-enhancing agents disclosed herein. Subtle changes in the images are detected more readily due to an increased contrast brought about by the ability of a contrast agent of the present invention to penetrate the areas of the endothelial layer of small blood vessels.

The method for detecting a diseased tissue comprises: (a) administering into a subject a predetermined dose of a conjugated polymer that comprises a polymer backbone chain conjugated to a plurality of molecules having at least a functionality such that the conjugated polymer achieves an extended conformation, the conjugated polymer further comprising at least an active agent that is capable of generating a signal detectable by a medical imaging technique; and (b) obtaining images of and said signal coming from the portion of the subject that is suspected to carry the disease before and after administering the conjugated polymer into the subject, a change in the images indicating a presence of the disease.

In one aspect, the method comprises: (a) administering into a subject a predetermined dose of at least an MRI contrast-enhancing agent that comprises an extended poly(amino acid) conjugated to chelator moieties that form coordination complexes with paramagnetic ions; and (b) obtaining MR images of and acquiring MR signals coming from the portion of the subject that is suspected to carry the disease before and after administering the MRI contrast-enhancing agent into the subject. When the disease, such as a tumor, is present the MR image acquired after the contrast agent has been administered into the subject shows an increased contrast and an increased MR signal compared to the image and signal acquired before administering the contrast-enhancing agent because there is an increased angiogenesis in the tissue. Such an increased contrast and increased M signal are a result of an increase in MR T₁ relaxation time. For example, an increase in the MR signal of 10 percent or more can signify the presence of the disease in the area under investigation.

In one aspect of the method, the MR contrast-enhancing agent is administered into the subject at a dose in the range from about 0.01 to about 0.05 moles Gd/kg of body weight of the subject. MR images and signals are acquired within 48 hours after the MR contrast agent is first administered into the subject. An MRI system that can be used for practicing a method of the present invention is disclosed in U.S. Pat. No. 6,235,264; which is incorporated herein by reference in its entirety. In one aspect of the present invention, a contrast-enhancing agent is administered intravenously into a subject. A contrast-enhancing agent can also be administered orally under appropriate circumstances.

In another aspect, the present invention provides a method for assessing an effectiveness of a prescribed regimen for treating a disease. The method comprises: (a) obtaining at least a base-line image of and acquiring a base-line signal from a portion of the subject that is suspected to carry the disease; (b) administering a first time into a subject a predetermined dose of a compound comprising an extended poly(amino acid) conjugated to molecules comprising p-SCN-Bz-DOTA, the extended poly(amino acid) further comprising an active agent that is capable of generating an enhanced level of the signal; (c) obtaining pre-treatment images of and acquiring pre-treatment signals coming from the same portion of the subject that is suspected to carry the disease after administering the predetermined dose of the compound into the subject; (d) treating a condition of the disease with the prescribed regimen; (e) administering a second time into the subject the predetermined dose of said compound; (f) obtaining post-treatment images of and acquiring post-treatment signals coming from the same portion of the subject as in step (c); and (g) comparing post-treatment images and post-treatment signals to pre-treatment images and pre-treatment signals to assess the effectiveness of the prescribed regimen.

In another aspect of the present invention, a method for assessing an effectiveness of a prescribed regimen for treating a disease comprises: (a) obtaining at least a base-line MR image of acquiring a base-line MR signal from a portion of the subject that is suspected to carry the disease; (b) administering a first time into a subject a predetermined dose of at least an MRI contrast-enhancing agent that comprises an extended poly(amino acid) conjugated to chelator moieties that form coordination complexes with paramagnetic ions; (c) obtaining pre-treatment MR images of and acquiring pre-treatment MR signals coming from the same portion of the subject that is suspected to carry the disease after administering the predetermined dose of the MRI contrast-enhancing agent into the subject; (d) treating a condition of the disease with the prescribed regimen; (e) administering a second time into the subject the predetermined dose of said at least an MRI contrast-enhancing agent; (f) obtaining post-treatment M images of and acquiring post-treatment MR signals coming from the same portion of the subject as in step (c); and (g) comparing post-treatment MR images and post-treatment MR signals to pre-treatment MR images and pre-treatment MR signals to assess the effectiveness of the prescribed regimen. A decrease in MR image contrast or MR signals during the course of the prescribed regimen indicates that the treatment has provided benefit. Such a decrease in MR image contrast or MR signal is a result of a decrease in the M T₁ relaxation time. The method further comprises repeating steps (e), (f), and (g) at predetermined time intervals during the course of treatment for atherosclerosis.

In one aspect of the method, the MR contrast-enhancing agent is administered into the subject at a dose in the range from about 0.01 to about 0.5 moles Gd/kg of body weight of the subject. MR images and signals are acquired within 48 hours after the dose of MR contrast agent is administered into the subject.

The prescribed regimen for treating the disease can be, for example, at least one of practicing a prescribed diet and exercise program, taking medication for treating a source of plaque deposit, or a combination thereof.

Although the foregoing methods are described in connection with the MRI technique, other medical imaging techniques can be used in place of MRI under appropriate circumstances if the contrast agent comprises an active agent that generates a signal detectable by the chosen imaging technique. Such other medical imaging techniques are disclosed herein above.

While various embodiments are described herein, it will be appreciated from the specification that various combinations of elements, variations, equivalents, or improvements therein may be made by those skilled in the art, and are still within the scope of the invention as defined in the appended claims.

1. A conjugated polymer comprising:
(a) a polymer backbone chain comprising a material selected from the group consisting of polylysine, polyhistidine, polyarginine, polyasparagine, polyglutamine, copolymers of at least two types of monomeric units selected from the group consisting of lysine, histidine, arginine, asparagines, and glutamine, copolymers of at least a first type of monomeric units selected from histidine, arginine, asparagines, and glutamine, and at least a second type of monomeric units selected from the group consisting of glutamic acid and aspartic acid;
(b) a plurality of molecules, each having at least a functionality, conjugated to the monomeric residues;
wherein at least a molecule having said at least a functionality is p-isothiocyanatobenzyl-1,4,7,10-tetraazacyclododecane-1,4,7,10-tetraacetic acid (“p-SCN-Bz-DOTA”).

2. The conjugated polymer of claim 1, wherein the polymer backbone chain is selected from the group consisting of polylysine, polyhistidine, polyarginine, and copolymers of at least two types of monomeric units selected from the group consisting of lysine, histidine, and arginine.

3. The conjugated polymer of claim 1, further comprising an active agent selected from the group consisting of diagnostic and therapeutic agents.

4. The conjugated polymer of claim 3, wherein the active agent is a diagnostic agent and comprises a material capable of generating a signal detectable by a technique selected from the group consisting of MRI, PET, SPECT, X-ray, and CT.

5. The conjugated polymer of claim 3, wherein the active agent is an MRI contrast-enhancing agent that comprises a paramagnetic ion.

6. The conjugated polymer of claim 5, wherein the paramagnetic ion is selected from the group gadolinium and dysprosium ions, and is bound to the p-SCN-Bz-DOTA molecules.

7. The conjugated polymer of claim 3, wherein the active agent is a diagnostic agent and comprises a material capable of generating positron emission.

8. The conjugated polymer of claim 7, wherein the material capable of generating positron emission comprises Cu-64, and is bound to the p-SCN-Bz-DOTA molecules.

9. The conjugated polymer of claim 4, wherein the active agent is a moiety comprising a material selected from the group consisting of 4-iodobenzamide and 4-fluorobenzamide, wherein the iodine atom is radioisotope I-124, and the fluorine atom is radioisotope F-18, and wherein the moiety is attached to at least a free amino group of an amino acid residue.

10. The conjugated polymer of claim 8, wherein the moiety is attached to at least a free amino group of a lysine residue.

11. The conjugated polymer of claim 3, wherein the active agent is a therapeutic agent and is selected from the group consisting of radioisotopes, drugs, toxins, fluorescent dyes activated by nonionizing radiation, hormones, hormone antagonists, receptor antagonists, enzymes, proenzymes activated by another agent, autocrine, and cytokine.

12. The conjugated polymer of claim 11, wherein the therapeutic agent is attached to at least a free amino group of an amino acid residue in the polymer backbone chain.

13. The conjugated polymer of claim 1, wherein the polymeric backbone chain comprises from about 50 to about 1500 monomeric residues.

14. The conjugated polymer of claim 1, wherein the polymeric backbone chain comprises from about 100 to about 650 monomeric residues.

15. The conjugated polymer of claim 1, wherein the plurality of molecules having said at least a functionality are conjugated to a fraction of the monomeric residues, and said fraction is in a range from about 50 to about 98 percent.

16. The conjugated polymer of claim 1, wherein the plurality of molecules having said at least a functionality are conjugated to a fraction of the monomeric residues, and said fraction is in a range from about 50 to about 90 percent.

17. The conjugated polymer of claim 1, wherein the plurality of molecules having said at least a functionality are conjugated to a fraction of the monomeric residues, and said fraction is in a range from about 50 to about 75 percent.

18. The conjugated polymer of claim 1, having a persistence length in a range from about 100 to about 600 angstroms.

19. A method for detecting a tissue carrying a disease, the method comprising:
(a) administering into a subject a predetermined dose of a conjugated polymer that comprises a polymer backbone chain conjugated to a plurality of molecules having at least a functionality such that the conjugated polymer achieves an extended conformation, the conjugated polymer further comprising at least an active agent that is capable of generating a signal detectable by a medical imaging technique; and
(b) obtaining images of and said signal coming from the portion of the subject that is suspected to carry the disease before and after administering the conjugated polymer into the subject, a change in the images indicating a presence of the disease.

20. The method of claim 19, wherein the conjugated polymer is an MRI contrast-enhancing agent that comprises an extended poly(amino acid) conjugated to chelator moieties that form coordination complexes with paramagnetic ions, wherein the chelator moieties comprise p-SCN-Bz-DOTA; and the medical imaging technique is MRI.

21. The method of claim 19, wherein the conjugated polymer is a PET imaging agent that comprises an extended poly(amino acid) conjugated to chelator moieties that form coordination complexes with Cu-64, wherein the chelator moieties comprise p-SCN-Bz-DOTA; and the medical imaging technique is PET.

22. The method of claim 19, wherein the conjugated polymer is a PET imaging agent that comprises an extended poly(amino acid) conjugated to a plurality of p-SCN-Bz-DOTA molecules, and further conjugated to at least a moiety labeled with an atom selected from the group consisting of I-124, F-18, Br-76, Br-77, and At-211.

23. The method of claim 19, wherein the medical imaging technique is CT.

24. A method for detecting a diseased tissue, the method comprising administering into a subject a predetermined dose of a conjugated polymer that comprises a polymer backbone chain conjugated to a plurality of molecules having at least a functionality such that the conjugated polymer achieves an extended conformation, the conjugated polymer further comprising at least an active agent that is capable of providing a beneficial effect on the diseased tissue.

25. A method for assessing an effectiveness of a prescribed regimen for treating a disease, the method comprising:
(a) obtaining at least a base-line image of and acquiring a base-line signal from a portion of the subject that is suspected to carry the disease, the signal being detectable by a medical imaging technique;
(b) administering a first time into a subject a predetermined dose of a compound comprising an extended poly(amino acid) conjugated to molecules comprising p-SCN-Bz-DOTA, the extended poly(amino acid) further comprising an active agent that is capable of generating an enhanced level of the signal;
(c) obtaining pre-treatment images of and acquiring pre-treatment signals coming from the same portion of the subject that is suspected to carry the disease after administering the predetermined dose of the compound into the subject;
(d) treating a condition of the disease with the prescribed regimen;
(e) administering a second time into the subject the predetermined dose of said compound;
(f) obtaining post-treatment images of and acquiring post-treatment signals coming from the same portion of the subject as in step (c); and
(g) comparing post-treatment images and post-treatment signals to pre-treatment images and pre-treatment signals to assess the effectiveness of the prescribed regimen.

26. The method according to claim 24, wherein the medical imaging technique is MRI.

27. The method according to claim 24, wherein the medical imaging technique is PET.

28. The method according to claim 24, wherein the medical imaging technique is CT.
